(19) United States
(12) Patent Application Publication
(10) Pub. No.: US 2008/0064709 A1
(43) Pub. Date: Mar. 13, 2008

(54) METHODS AND COMPOSITIONS FOR THE TREATMENT OF VASCULAR DEPRESSION

(75) Inventor: Ranga Krishnan, Chapel Hill, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Duke University, Durham, NC (US)

(21) Appl. No.: 11/852,589

(22) Filed: Sep. 10, 2007

Related U.S. Application Data
(60) Provisional application No. 60/843,426, filed on Sep. 11, 2006.

(51) Int. Cl. A61K 31/522; A61P 25/24

(52) U.S. Cl. 514/263.3

(57) ABSTRACT

Methods and compositions are provided for treating vascular depression. The methods involve administering to a subject in need thereof a xanthine derivative in a therapeutically effective amount to treat vascular depression, particularly the xanthine derivatives pentoxifylline or propentofylline. The methods may further include administration of an additional therapeutic agent in combination with the xanthine derivative selected from the group consisting of a selective serotonin reuptake inhibitor (SSRI), a serotonin-norepinephrine reuptake inhibitor (SNRI), and a drug used in the treatment of cerebrovascular disease. Compositions of the invention include pharmaceutical compositions and kits for treating vascular depression in a subject in need thereof that include therapeutically effective amounts of a xanthine derivative and an additional therapeutic agent selected from the group consisting of an SSRI, an SNRI, and a drug used in the treatment of cerebrovascular disease.

### CROSS-REFERENCE TO RELATED APPLICATIONS

This claims the benefit of U.S. Provisional Patent Application No. 60/843,426, filed Sep. 11, 2006, which is hereby incorporated by reference in its entirety.

### FIELD OF THE INVENTION

The present invention relates, in general, to methods and compositions for treating vascular depression, particularly methods and compositions comprising xanthine derivatives.

### BACKGROUND OF THE INVENTION

Older individuals often demonstrate age-related deficits in a variety of cognitive domains, including psychomotor speed, memory retrieval, working memory, loss of inhibitory control, and alterations in attention (Andres and Van der Linden, J. Gerontol. B. Psychol. Sci. Soc. Sci. 55:P373-P380 (2000)). This has led to the theory that frontal lobe impairment may be related to the phenomenon of age-related cognitive decline (West, Psychol. Bull. 120:272-292 (1996)). These cognitive functions all localize to a series of parallel prefrontal pathways (Tekin and Cummings, J. Psychosom. Res. 53:647-654 (2002)) with similar anatomic features. They are tightly segregated and start in the frontal lobe, project to the ventral striatum, pass to the globus pallidus and substantia nigra, and through the thalamus connect back to the frontal lobe. Three of these circuits exhibit behavioral correlates: executive dysfunction (dorsolateral prefrontal circuit), apathy (anterior cingulate circuit), and mood lability and disinhibition (orbitofrontal circuit). Damage to or lesions that occur anywhere along the circuit may result in similar clinical findings. Subcortical vascular disease, which may include subcortical ischemia leading to silent lacunar infarctions or hyperintense lesions (areas of increased signal intensity in the periventricular and deep white matter seen best on T2-weighted magnetic resonance images), is one possible cause of disruption to these circuits

Subcortical ischemic disease is common even in otherwise neurologically normal elderly patients (Bryan et al., AJNR Am. J. Neuroradiol. 20:1273-1280; Krishnan, Biol. Psychiatry 43:705-712 (1998); Krishnan et al., Biol. Psychiatry 55:390-397 (2004); Taylor et al., Psychiatry Res. 139:1-7 (2005)). However, vascular changes may also be associated with cognitive impairment not reaching the severity of dementia, or with other psychiatric syndromes, including psychosis (Breitner et al., Biol. Psychiatry 28:266-274 (1990); Tonkonogy and Geller, Neuropsychiatry Neuropsychol. Behav. Neurol. 12:230-235 (1999)) and bipolar disorder (McDonald et al., Biol. Psychiatry 45:965-971 (1999)). Moreover, white matter hyperintense lesions are associated with motor deficits, such as gait dysfunction (Whitman, Neurology 57:990-994 (2001)) and urinary incontinence (Sakakibara et al., J. Neurol. Neurosurg. Psychiatry 67:658-660 (1999); Tarvonen-Schroder et al., J. Neurol. Neurosurg. Psychiatry 60:431-436 (1996)), presumably through the failure to inhibit bladder contractions. Thus, one etiology (cerebrovascular disease) may have a variety of clinical presentations depending on how it affects motor, cognitive, and emotional neural circuits. Additionally, this is not a static process; progression of subcortical ischemic disease has been associated with poorer longer-term antidepressant treatment outcomes (Taylor et al., Biol. Psychiatry 53:144-149 (2003)).

Acknowledging that subcortical ischemic disease may have various clinical presentations, there is considerable evidence that it contributes to the risk of developing depression in late life. Subcortical lesions are more common in elderly depressed than healthy elderly control subjects (Coffey et al., Arch. Gen. Psychiatry 50:7-16 (1993); de Groot et al., Arch. Gen. Psychiatry 57:1071-1076 (2000); Greenwald et al., Am. J. Psychiatry 153:1212-1215 (1996); Krishnan et al., Eur. Arch. Psychiatry Clin. Neurosci. 243:41-46 (1993); Kumar et al., Neuropsychopharmacology 22:264-274 (2000); Taylor et al.; Psychiatry Res. 139:1-7(2005); Tupler et al., J. Psychosom. Res. 53:665-676 (2002)), and are more common in late-onset than early-onset elderly depressed subjects (Dahabra et al., Biol. Psychiatry 44:34-46 (1998); de Groot et al., Arch. Gen. Psychiatry 57:1071-1076 (2000); Figiel et al., Neurobiol. Aging 12:245-247 (1991); Krishnan et al., Am. J. Psychiatry 154:497-501 (1997); Lavretsky et al., Am. J. Geriatr. Psychiatry 6:248-256 (1998); Murata et al., Int. J. Geriatr. Psychiatry 16:11291135 (2001); Salloway et al., Neurology 46:1567-1574 (1996); Tupler et al., J. Psychosom. Res. 53:665-676 (2002). This difference has also been demonstrated in community populations with depressive symptoms (Steffens et al., Am. J. Geriatr. Psychiatry 7:34-40 (1999); Steffens et al., Stroke 30:2159-2166 (1999)), extending the finding beyond the artificial symptom cutpoints that define Major Depression criteria.

Subcortical lesions are associated with poor antidepressant outcomes (O'Brien et al., BMJ 317:982-984 (1998); Papakostas et al., Psychiatry Res. 140:301-307 (2005); Simpson et al., Psychol. Med. 28:1015-1026 (1998)), and larger studies have shown subcortical lesions may be associated with persistence or worsening of depressive symptoms over time (Steffens et al., Stroke 33: 16361644 (2002)), and that poorer depression course over time is associated with greater increases in subcortical white matter lesion volume (Taylor et al., Biol. Psychiatry 53:144-149 (2003)). There may also be a biological gradient, wherein lesions contributing to depression may need to occur in specific regions (Greenwald et al., Stroke 29:613-617 (1998), MacFall et al., Biol. Psychiatry 49:803-806 (2001); MacFall et al., Neuropsychopharmacology 31:1500-1507 (2006); Taylor et al., Prog. Neuropsychopharmacol. Biol. Psychiatry 27:539-544 (2003)). These lesions are clearly related to medical comorbidity, particularly cerebrovascular risk factors. Greater severity of subcortical lesions are associated with factors such as hypertension (Dufouil et al., Neurology 56:921-926 (2001); Fukuda and Kitani, Stroke 26:1593-1597 (1995); Liao et al., Stroke 27:2262-2270 (1996); Schmidt et al., Neurology 53:132-139 (1999); Veldink et al., Neurology 51:319-320 (1998), as well as metabolic factors including hypofolatemia (Iosifescu et al., Psychiatry Res. 140:291-299 (2005)). The ischemic nature of these lesions, has also been shown in post-mortem studies (Thomas et al., Arch. Gen. Psychiatry 59:785-792 (2002)).

The present invention provides a method of treating vascular depression (late-life depression) associated with subcortical ischemic disease.

### SUMMARY OF THE INVENTION

Methods and compositions for treating vascular depression are provided. The methods involve administering to a subject in need thereof a xanthine derivative in a therapeutically effective amount to treat vascular depression. Exemplary xanthine derivatives for use in these methods include, but are not limited to, pentoxifylline(1-(5-oxohexyl)-3,7-dimethylxanthine) and propentofylline(1-(5-oxohexyl)-3-methyl-7-n-propylxanthine). The methods may further include administration of an additional therapeutic agent in combination with the xanthine derivative. In one such embodiment, the additional therapeutic agent is a drug used in the treatment of cerebrovascular disease. In another embodiment, the additional therapeutic agent is a selective serotonin reuptake inhibitor (SSRI) or a serotonin-norepinephrine reuptake inhibitor (SNRI).

Compositions of the invention include pharmaceutical compositions and kits for treating vascular depression in a subject in need thereof. In some embodiments, the pharmaceutical compositions and kits include therapeutically effective amounts of a xanthine derivative and an additional therapeutic agent selected from the group consisting of an SSRI, an SNRI, and a drug used in the treatment of cerebrovascular disease.

### DETAILED DESCRIPTION OF THE INVENTION

Overview

The present invention provides methods and compositions for treating vascular depression in a subject in need thereof. The methods comprise administering to a subject in need thereof a xanthine derivative in a therapeutically effective amount to treat vascular depression. In some embodiments, the methods comprise administration of a xanthine derivative in combination with at least one other therapeutic agent. Pharmaceutical compositions and kits for treating vascular depression in a subject in need thereof are also provided. These pharmaceutical compositions and kits comprise therapeutically effective amounts of a xanthine derivative and one or more additional therapeutic agent. In one such embodiment, the pharmaceutical compositions and kits include a xanthine derivative and a drug used in the treatment of cerebrovascular disease. In another embodiment, the pharmaceutical compositions and kits include a xanthine derivative and a selective serotonin reuptake inhibitor (S SRI) or a serotonin-norepinephrine reuptake inhibitor (SNRI). In particular embodiments of the methods and compositions of the invention, xanthine derivatives include but are not limited to pentoxifylline(1-(5-oxohexyl)-3,7-dimethylxanthine) and propentofylline(1-(5-oxohexyl)-3-methyl-7-n-propylxanthine).

Vascular depression is a condition that typically affects the elderly and is associated with cerebrovascular disease that can lead to small vascular lesions in the brain (also referred to herein as small vessel disease). Patients with vascular depression form a distinct clinical population characterized as having greater cognitive dysfunction, disability, retardation, and lack of insight compared to patients with nonvascular depression, as well as less agitation and depressive ideation (Alexopoulos et al., Am. J. Psychiatry 154:562-565 (1997)). The disease is progressive and can lead to dementia.

The present invention results, at least in part, from the recognition that xanthine derivatives such as pentoxifylline and derivatives, analogs, and/or metabolites thereof can be used to treat patients with vascular depression and associated small vessel disease. The precise mechanism of action of pentoxifylline is unclear but, without being bound by any particular theory or mechanism of action, pentoxifylline is believed to effect white blood cell function and haemorrheological parameters. It is also thought that pentoxifylline may be useful for increasing cerebral blood flow. In a double-blinded, placebo-controlled, multicenter study, treatment with pentoxifylline was found to improve intellectual and cognitive function for patients with multi-infarct dementia (European Pentoxifylline Multi-Infarct Dementia Study, Eur. Neurol. 36:315-321 (1996)). However, the effect of xanthine derivatives on vascular depression has not previously been described.

Methods

The present invention provides a method for treating vascular depression by administering to a subject in need thereof a therapeutically effective amount of a xanthine derivative, alone or in combination with an additional therapeutic agent. In one embodiment, the xanthine derivatives for use in the methods and compositions of the present invention comprise a compound of Formula I 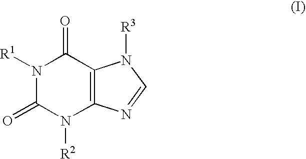
or a physiologically tolerable salt of the compounds of the Formula I, where R¹ is

a) an oxoalkyl group having 3 to 8 carbon atoms, whose carbon chain can be straight-chain or branched;

b) a hydroxyalkyl group having 1 to 8 carbon atoms, whose carbon chain can be straight-chain or branched and whose hydroxyl group is a primary, secondary or tertiary alcohol function; or

c) an alkyl group having 1 to 6 carbon atoms, whose carbon chain can be straight-chain or branched;

where R² is

a) a hydrogen atom; or

b) an alkyl group having 1 to 4 carbon atoms, whose carbon chain can be straight-chain or branched; and

where R³ is

a) a hydrogen atom;

b) an alkyl group having 1 to 6 carbon atoms, whose carbon chain can be straight-chain or branched;

c) an alkyl group having 1 to 6 carbon atoms, whose carbon chain is interrupted by an oxygen atom; or

d) an oxoalkyl group having 3 to 8 carbon atoms, whose carbon chain can be straight-chain or branched.

Preferably, compounds of the Formula I are used wherein

R¹ is an oxoalkyl group having 4 to 6 carbon atoms, whose carbon chain is straight-chain, or an alkyl group having 3 to 6 carbon atoms;

R² is an alkyl group having 1 to 4 carbon atoms; and

R³ is an alkyl group having 1 to 4 carbon atoms or an oxoalkyl having 3 to 6 carbon atoms.

In one embodiment, xanthine derivatives according to Formula I for use in the methods and compositions of the present invention include: 1-(5-oxohexyl)-3,7-dimethylxanthine(pentoxifylline); 1-(5-(R)-hydroxyhexyl)-3,7-dimethylxanthine(lisofylline); 1-(5-(S)-hydroxyhexyl)-3,7-dimethylxanthine; 1-(5-oxohexyl)-3-methyl-7-n-propylxanthine(propentofylline); 1-(5-hydroxy-5-methyl-hexyl)-3-methylxanthine; 1-(5-hydroxy-5-methyl-hexyl)-3,7-dimethylxanthine(torbafylline); 7-(ethoxymethyl-1-(5-hydroxy-5-methylhexyl)-3-methylxanthine; 1-(5-oxohexyl)-3,7-dimethylxanthine; 7-(2-oxopropyl)-1,3-di-n-butylxanthine; and 1-hexyl-3,7-dimethylxanthine. Suitable physiologically tolerable salts of the xanthine derivatives of Formula I are, for example, alkali metal, alkaline earth metal or ammonium salts, including those of physiologically tolerable organic ammonium bases.

The compounds of the Formula I may be prepared under standard conditions in a known manner. Processes for preparing the compounds according to Formula I are described, for example, in U.S. Pat. Nos. 4,289,776; 4,833,146; and 3,737,433. The starting substances of the reactions are known to those of one of ordinary skill in the art or can be easily prepared by methods known from the literature.

In a particular embodiment, the xanthine derivatives according to Formula I for use in the methods and compositions of the present invention are 1-(5-oxohexyl)-3,7-dimethylxanthine(pentoxifylline) or 1-(5-oxohexyl)-3-methyl-7-n-propylxanthine(propentofylline).

The chemical structure for pentoxifylline is depicted below:

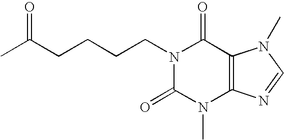

The chemical structure for propentofylline is depicted below:

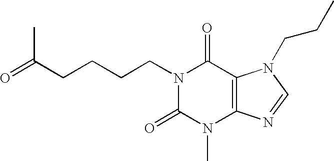

The vasodilatory effects of pentoxifylline, and certain derivatives thereof, are described in U.S. Pat. No. 3,737,433. A metabolite of pentoxifylline, 1(5-hydroxyhexyl)-3,7dimethylxanthine, is described in U.S. Pat. Nos. 4,515,795 and 4,576,947 as increasing cerebral blood flow, as are tertiary alcohol analogs of xanthine (including those described in U.S. Pat. Nos. 4,833,146 and 5,039,666). Other metabolites of pentoxifylline are reported in Davis et al. App. Env. Micro. 48:327 (1984). Still other derivatives, metabolites, and/or analogs of pentoxifylline include those referenced in U.S. Pat. No. 5,795,897, as well as prodrug forms or salts (e.g., a maleate salt) thereof as described, for example, in U.S. Pat. Nos. 4,975,432 and 5,118,500. All of the above-referenced derivatives, metabolites, analogs, prodrugs, and salts of pentoxifylline are contemplated for use within the methods and compositions of the present invention.

Still further xanthine derivatives that can be utilized in the practice of this invention include those set forth in U.S. Pat. Nos. 4,108,995; 4,242,345; 4,289,776; 4,291,037; 4,515,795; 5,407,815; 5,478,831; 6,037,347 and U.S Pat. App. Pub. Nos. 20050191375 and 20070185213. In one embodiment, the xanthine derivative for use in the methods and compositions of the present invention is a methyl xanthine.

Within the context of the present invention, a “subject” is a patient diagnosed by a physician as having vascular depression using criteria known in the art. Thus, the terms “subject” and “patient” are used interchangeably herein. Although a diagnosis of vascular depression may include an assessment of vascular lesions in the brain via neuroimaging (e.g., using MRI), vascular depression may also be diagnosed in the absence of imaging where individuals have experienced a first onset of depression at 60 years of age or older (late-onset depression). Criteria for diagnosing vascular depression include, but are not limited to, the methodology of Krishnan et al., Am. J. Psychiatry 154:497-501 (1997), and/or the methodology of Alexopoulos et al., Am. J. Psychiatry 154:562-565 (1997). For example, Krishnan et al. identified patients with vascular dementia as those individuals with a diagnosis of major depressive disorder who had no co-morbid dementia or other major neurological disorders, whose depression was not secondary to medications (e.g., steroids) or medical illnesses, who had low-to-moderate scores on the Mini-Mental State scale, and who were identified as having vascular lesions in the brain using MRI (Am. J. Psychiatry 154:497-501 (1997)). Alexopoulos et al. identified patients with vascular dementia as those individuals with a first onset of depression at 60 years of age or older who had no co-morbid dementia or other major neurological or psychiatric disorders prior to the first depressive episode, who did not suffer from major medical illnesses such as metastatic cancer or decompensated cardiac, hepatic, or renal failure, who had low-to-moderate scores on the Mini-Mental State scale, and who had a vascular score of 1-4 on the Cumulative Illness Rating Scale—Geriatrics (Am. J. Psychiatry 154:562-565 (1997); Geldmacher and Whitehouse “Multi infarct dementia” in Psychopharmacology: The Fourth Generation of Progress. Bloom and Kupfer Eds. New York, Raven Press, 1993, pp. 1513-1520). Although exemplary, other methods and criteria for diagnosing vascular dementia in addition to those of Krishnan et al. and Alexopoulos et al. may be used in the context of the present invention, and are well-known to one of skill in the art.

As described above, the present invention provides a method for treating vascular depression by administering to a subject in need thereof a therapeutically effective amount of a xanthine derivative. By “therapeutically effective amount” is meant the concentration of a xanthine derivative or additional therapeutic agent that is sufficient to elicit a therapeutic effect. Thus, the concentration of a xanthine derivative or additional therapeutic agent in accordance with the present invention is effective in the treatment or prevention of one or more symptoms or diagnostic criteria for vascular depression as described elsewhere herein (i.e., such symptoms or diagnostic criteria can be minimized or eliminated and/or the likelihood of relapse can be reduced).

For use within the methods of the present invention, therapeutically effective doses of xanthine derivatives may be administered using any acceptable method known in the art. Preferred administration routes include oral, parenteral (e.g., intraperitoneal, intravenous, subcutaneous, or intramuscular), transdermal (e.g., using a patch) and sublingual. Optimum doses can be readily established by one skilled in the art (e.g., 25-800 mg three times per day).

Dosage levels are based upon a body weight of approximately 70 kg. It will be understood, however, that the specific dose level and frequency of dosage for any particular subject may be varied and will depend upon a variety of factors including body weight, age, general health, sex, and diet of the subject, the metabolic stability and length of action of the administered compound, mode and time of administration, rate of excretion, drug combination, formulation, and severity of the vascular depression and/or cerebrovascular disease.

Thus, in one embodiment, a therapeutically effective amount of a xanthine derivative or additional therapeutic agent within the methods and compositions of the present invention is administered in a dose of from about 1 μg to about 50 mg per kg of body weight, from about 0.01 mg to about 40 mg per kg of body weight, from about 0.05 mg to about 30 mg per kg of body weight, from about 0.1 mg to about 20 mg per kg of body weight, from about 0.1 mg to about 10 mg per kg of body weight, from about 0.5 mg to about 5 mg per kg of body weight, from about 1 mg to about 50 mg per kg of body weight, from about 1 mg to about 40 mg per kg of body weight, from about 1 mg to about 30 mg per kg of body weight, from about 1 mg to about 20 mg per kg of body weight, and from about 1 mg to about 10 mg per kg of body weight. Alternatively, the amount of a xanthine derivative or additional therapeutic agent administered to achieve a therapeutically effective dose is about or at least about 1 μg, 10 μg, 100 μg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, or 50 mg per kg of body weight or greater.

In another embodiment, a therapeutically effective amount of a xanthine derivative or additional therapeutic agent within the methods and compositions of the present invention is administered orally in a total daily dose of between about 100 mg to about 1200 mg, about 100 mg to about 2400 mg, about 500 mg to about 1200 mg, about 500 mg to about 2400 mg, or about 600 mg to about 1800 mg. In such embodiments, the total daily dose is in an amount of about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200 mg, about 2250 mg, about 2300 mg, about 2350 mg, or about 2400 mg. Furthermore, the xanthine derivative or additional therapeutic agent may be administered in a single dose or multiple doses to achieve a total daily dose as described above. For example, in one embodiment, the xanthine derivative or additional therapeutic agent is administered two, three, or more times a day in individual doses of about 25 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg.

In particular embodiments, the xanthine derivative is pentoxifylline or propentofylline and is administered orally. For example, pentoxifylline can be administered in tablet or capsule form under the trade name Trental® (Aventis Pharmaceuticals, Bridgewater, N.J.). In one embodiment, the xanthine derivative is pentoxifylline and is administered orally in a total daily dosage of between about 400 mg and about 1200 mg, e.g. about 400 mg three times a day. In another embodiment, the xanthine derivative is propentofylline and is administered orally in a total daily dosage of between about 200 mg and about 600 mg, e.g., about 200 mg three times a day.

As used herein, the term “about,” when referring to a value is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

As described above, the present invention also provides a method for treating vascular depression by administering to a subject in need thereof a therapeutically effective amount of a xanthine derivative in combination with an additional therapeutic agent. In one embodiment, the additional therapeutic agent includes drugs used in the treatment of cerebrovascular disease, including but not limited to anticholesterolemic and antiplatelet agents, free radical scavengers, calcium channel blockers, glutamate N-methyl-D-aspartic acid (NMDA) receptor antagonists, gangliosides, aminosteroids, and amphetamines (Alexopoulos et al., Am. J. Psychiatry 154:562-565 (1997)).

Anticholesterolemics include a wide variety of lipid lowering agents, including but not limited to bile acid sequestrants, HMG-CoA reductose inhibitors, and statins (e.g., lovastatin, provastatin and the like).

Anticoagulant drugs are drugs used to prevent clot formation or to prevent a clot that has formed from enlarging. Examples of anticoagulant drugs fall into three groups: 1) inhibitors of clotting factor synthesis (e.g., warfarin); 2) inhibitors of thrombin (e.g., heparin and lepirudin); and 3) antiplatelet drugs (e.g., aspirin, ticlopidine, clopidogrel, tirofiban, and eptifibatide).

Free-radical scavengers are agents that sequester free radicals so they do not initiate oxidative reactions that can lead to cellular damage. These free-radical scavengers can be naturally occurring substances such as beta-carotene, vitamins C and E, estrogen, and Ginkgo biloba, or can be synthetically prepared substances such as selegiline, lazabemide, and tenilsetam.

Calcium channel blockers are drugs that relax blood vessels and increase the supply of blood and oxygen to the heart. Exemplary calcium channel blockers include, but are not limited to, nisoldipine, nifedipine, nicardipine, bepridil, isradipine, nimodipine, felodipine, amlodipine, diltiazem, and verapamil.

Antagonists of NMDA subtype of glutamate receptor cinlude, but are not limited to, amantadine, dextromethorphan, dextrophan, dizocilpine, ibogaine, ketamine, nitrous oxide, phencyclidine, riluzole, tiletamine, aptiganel, memantine, remacimide, 7-chlorokynurenate, DCKA (5,7-dichlorokynurenic), AP7 (2-amino-7-phosphonoheptanoic acid), APV (R-2-amino-5-phosphonopentanoate), and CPPene (3-[(R)-2-carboxypiperazin-4-yl]-prop-2-enyl-1-phosphonic acid).

Gangliosides are glycosphingolipids that contain an oligosaccharide moiety to which may be attached one or more sialic acid groups. According to the nomenclature proposed by Svennerholm (J. Neurochem., 10:613 (1963)) the various gangliosides are designated by the letter G followed by one of four letters, M, D, T or Q depending on whether the ganglioside is a mono-, di-, tri- or tetra-sialo-ganglioside. These letters are followed by numerical indices allowing recognition of gangliosides containing the same quantity of sialic acid but having different chromatographic mobilities. Examples of gangliosides are well known in the art, and include GD1a, GD1b, GD2, GD3, GM1, GM2, GM3, and GT1b (see, e.g., Svennerholm et al., Q. Rev. Biol., 56:329 (1981)).

Aminosteroids are a group of drugs with a similar structure based on a steroid nucleus. Examples of aminosteroids include, but are not limited to, rocuronium, vecuronium, and pancuronium.

Amphetamines are synthetic psychostimulant drugs, and include both amphetamine and amphetamine derivatives, including but not limited to, methamphetamine, ethylamphetamine, dimethylamphetamine, PPMA, N-hydroxyamphetamine, N-hydroxymethamphetamine, phenethylamine (PEA), (+) cathine, (−) cathinone, methcathinone, amfepramone, amphetaminil, methylenedioxy-amphetamines (e.g., MDA and MDMA), and ring and side-chain substituted amphetamines (e.g., diethoxybromoamphetamine).

In another embodiment, the present invention relates to a method for treating vascular depression by administering to a subject in need thereof a therapeutically effective amount of a xanthine derivative in combination with a selective serotonin reuptake inhibitor (SSRI) or serotonin-norepinephrine reuptake inhibitor (SNRI) antidepressant agent. Animal studies suggest that some antidepressants promote neurological recovery after ischemic lesions but that other antidepressants, as well as haloperidol, phenytoin, and benzodiazepines, inhibit recovery (Alexopoulos et al., Am. J. Psychiatry 154:562-565 (1997); Goldstein, J. Neurol. Rehab. 5:129-140 (1991); Boyerson et al., Arch. Neurol. 51:405-414 (1994)). In particular, SSRIs and SNRIs have been shown to be effective in the treatment of depression in elderly patients (Ravindran et al., Geriatrics Aging 8:20-27(2005)). SSRIs and SNRIs increase the extracellular level of serotonin (SSRIs) or both serotonin and norepinephrine (SNRIs) available to bind to postsynaptic receptors by inhibiting reuptake into the presynaptic cell.

Accordingly, in one embodiment, the present invention relates to a method for treating vascular depression by administering to a subject in need thereof a therapeutically effective amount of a xanthine derivative in combination with an SSRI. In one embodiment, the method comprises treating vascular depression by administering to a subject in need thereof a xanthine derivative according to Formula I, particularly pentoxifylline or propentofylline, in combination with an SSRI. In another embodiment, the SSRI to be administered in combination with a xanthine derivative is selected from the group consisting of citalopram, escitalopram, sertraline, paroxetine, fluoxetine, fluvoxamine, and dapoxetine. In a preferred embodiment, the SSRI to be administered in combination with a xanthine derivative is citalopram or escitalopram.

As described above, the specific dose level and frequency of dosage of SSRI to be administered in combination with a xanthine derivative may be varied for any particular subject and will depend upon a variety of factors. Exemplary dosage ranges for additional therapeutic agents within the methods of the invention are provided elsewhere wherein. In particular embodiments, the methods of the present invention comprise administration of a therapeutically effective amount of a xanthine derivative (particularly a compound according to Formula I, more particularly pentoxifylline or propentofylline) in combination with a total daily dose of about 20 mg to about 30 mg of citalopram, about 10 mg to about 20 mg of escitalopram, about 25 to about 200 mg of sertraline, about 10 mg to about 50 mg of paroxetine, about 5 mg to about 60 mg of fluoxetine, about 100 mg to about 300 mg of fluvoxamine, and about 30 mg to about 60 mg of dapoxetine. The SSRI may be administered in a single dose or multiple doses to achieve a total daily dose as described above.

Accordingly, in one embodiment, the present invention relates to a method for treating vascular depression by administering to a subject in need thereof a therapeutically effective amount of a xanthine derivative in combination with an SNRI. In one embodiment, the method comprises treating vascular depression by administering to a subject in need thereof a xanthine derivative according to Formula I, particularly pentoxifylline or propentofylline, in combination with an SNRI. In another embodiment, the SNRI to be administered in combination with a xanthine derivative is selected from the group consisting of venlafaxine, desvenlafaxine, nefazodone, milnacipran, desipramine, and duloxetine. In a preferred embodiment, the SSRI to be administered in combination with a xanthine derivative is venlafaxine.

As described above, the specific dose level and frequency of dosage of SNRI to be administered in combination with a xanthine derivative may be varied for any particular subject and will depend upon a variety of factors. Exemplary dosage ranges for additional therapeutic agents within the methods of the invention are provided elsewhere wherein. In particular embodiments, the methods of the present invention comprise administration of a therapeutically effective amount of a xanthine derivative (particularly a compound according to Formula I, more particularly pentoxifylline or propentofylline) in combination with a total daily dose of about 75 mg to about 225 mg of venlafaxine, about 200 mg to about 600 mg of desvenlafaxine, about 200 mg to about 600 mg of nefazodone, about 50 mg to about 100 mg of milnacipran, about 75 mg to about 300 mg of desipramine, and about 40 mg to about 60 mg of duloxetine. The SNRI may be administered in a single dose or multiple doses to achieve a total daily dose as described above.

Where the xanthine derivative is administered in combination with an additional therapeutic agent to treat vascular depression in a patient in need thereof, the administration of these agents can occur concurrently (simultaneously) or sequentially (consecutively) in any order. For concurrent administration of multiple therapeutic agents (for example, a xanthine derivative such as pentoxifylline or propentofylline in combination with an SSRI or SNRI), the multiple agents may be formulated either within a single pharmaceutical composition or within separate pharmaceutical compositions (for example, one formulation comprising the xanthine derivative and one formulation comprising the SSRI or SNRI). For sequential administration, each therapeutic agent can be formulated in its own pharmaceutical composition, each of which is to be administered sequentially, in any order.

Where the xanthine derivative is administered in combination with an SSRI or SNRI, the xanthine derivative may be administered as an add-on therapy for patients who are already being treated with an SSRI or SNRI. Thus in one embodiment, the xanthine derivative (such as pentoxifylline or propentofylline) may be used to augment existing SSRI or SNRI treatment of vascular depression in a patient in need thereof. As described above, administration of these agents can occur concurrently (simultaneously) or sequentially (consecutively) in any order.

The toxicity and therapeutic efficacy of xanthine derivatives or additional therapeutic agents within the methods and compositions of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. The dosage lies preferably within a range of circulating concentrations that include the ED₅₀ with little or no toxicity, and can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Compositions

The present invention also relates to a pharmaceutical composition for treating vascular depression in a subject in need thereof, comprising a xanthine derivative in combination with an additional therapeutic agent comprising an SSRI, SNRI, or an agent that is used in the treatment of cerebrovascular disease, together with a pharmaceutically acceptable carrier. In such pharmaceutical compositions, the xanthine derivative and additional therapeutic agent are in therapeutically effective amounts for treating vascular depression in a subject in need thereof. As used herein the term “pharmaceutically acceptable carrier” includes solvents, dispersion media, coatings, antibacterial and antifungal agents, isotonic and absorption delaying agents, and the like, compatible with pharmaceutical administration.

Thus in one embodiment, the present invention provides a pharmaceutical composition for treating vascular depression in a subject in need thereof comprising a xanthine derivative in combination with an SSRI. In one embodiment, xanthine derivative is a compound according to Formula I. In another embodiment, the xanthine derivative is pentoxifylline or propentofylline. In yet another embodiment, the SSRI is selected from the group consisting of citalopram, escitalopram, sertraline, paroxetine, fluoxetine, fluvoxamine, and dapoxetine. In another embodiment, the SSRI is citalopram or escitalopram.

In another embodiment, the present invention provides a pharmaceutical composition for treating vascular depression in a subject in need thereof comprising a xanthine derivative in combination with an SNRI. In one embodiment, xanthine derivative is a compound according to Formula I. In another embodiment, the xanthine derivative is pentoxifylline or propentofylline. In yet another embodiment, the SNRI is selected from the group consisting of venlafaxine, desvenlafaxine, nefazodone, milnacipran, desipramine, and duloxetine. In another embodiment, the SNRI is venlafaxine.

In another embodiment, the present invention provides a pharmaceutical composition for treating vascular depression in a subject in need thereof comprising a xanthine derivative in combination with an agent that is used in the treatment of cerebrovascular disease. In one embodiment, xanthine derivative is a compound according to Formula I. In another embodiment, the xanthine derivative is pentoxifylline or propentofylline. In yet another embodiment, the agent that is used in the treatment of cerebrovascular disease is selected from the group consisting of anticholesterolemic and antiplatelet agents, free radical scavengers, calcium channel blockers, glutamate N-methyl-D-aspartic acid (NMDA) receptor antagonists, gangliosides, aminosteroids, and amphetamines.

As one of ordinary skill in the art will appreciate, the presently disclosed pharmaceutical compositions are formulated to be compatible with their intended route of administration (e.g., oral, transdermal, sublingual, and parenteral, including intraperitoneal, intravenous, subcutaneous, or intramuscular administration). Suitable formulations and their appropriate carrier vehicles are described, for example, in Remington's Pharmaceutical Sciences (18^{th} ed.; Mack Publishing Company, Eaton, Pa., 1990), herein incorporated by reference.

A number of formulations exist relating to dosage forms for pentoxifylline, e.g., U.S. Pat. Nos. 4,327,725; 4,612,008; 4,765,989; 4,783,337; 5,532,003; and 5,603,954, and the present invention contemplates adapting such pharmaceutical compositions to incorporate an additional therapeutic agent comprising an SSRI, SNRI, or an agent that is used in the treatment of cerebrovascular disease.

Oral formulations generally include an inert diluent or an edible carrier. For the purpose of oral therapeutic administration, the xanthine derivative and additional therapeutic agent can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches, and the like can contain any of the following ingredients, or compounds of a similar nature: a binder, such as microcrystalline cellulose, gum tragacanth or gelatin; an excipient, such as starch or lactose, a disintegrating agent, such as alginic acid, Primogel, or corn starch; a lubricant, such as magnesium stearate or Sterotes; a glidant, such as colloidal silicon dioxide; a sweetening agent, such as sucrose or saccharin; or a flavoring agent, such as peppermint, methyl salicylate, or orange flavoring. Formulations for oral delivery can advantageously incorporate agents to improve stability within the gastrointestinal tract and/or to enhance absorption.

Solutions or suspensions used for parenteral (e.g., intravenous), intramuscular, intradermal, or subcutaneous application can include the following components: a sterile diluent such as water for injection, saline solution, fixed oils, polyethylene glycols, glycerine, propylene glycol or other synthetic solvents; antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates; and agents for the adjustment of tonicity, such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The composition should be sterile and should be fluid to the extent that easy syringability exists. Preferred pharmaceutical compositions are stable under the conditions of manufacture and storage and should be preserved against the contaminating action of microorganisms, such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants.

Systemic administration of the presently disclosed compositions also can be by transmucosal or transdermal means. For transmucosal or transdermal administration, penetrants appropriate to the barrier to be permeated are used in the composition. Such penetrants are generally known in the art, and include, for example, for transmucosal administration, detergents, bile salts, and fusidic acid derivatives. Transmucosal administration can be accomplished through the use of nasal sprays or suppositories (urethral or rectal). For transdermal administration, the active compounds are formulated into ointments, salves, gels, or creams as generally known in the art.

The presently disclosed compositions also can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials also can be obtained commercially from Alza Corporation and Nova Pharmaceuticals, Inc. Liposomal suspensions (including liposomes targeted to infected cells with monoclonal antibodies to viral antigens) also can be used as pharmaceutically or cosmetically acceptable carriers. Such suspensions can be prepared according to methods known to those skilled in the art, for example, as described in U.S. Pat. No. 4,522,811, which is incorporated herein by reference in its entirety.

It is advantageous to formulate oral or parenteral formulations in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

The present invention also relates to a packaged kit that contains a xanthine derivative in combination with an additional therapeutic agent comprising an SSRI, SNRI, or an agent that is used in the treatment of cerebrovascular disease, each in therapeutically effective amounts to treat vascular depression in a subject in need thereof. The packaged kit will also include a container for housing the active agents during storage and prior to use, and instructions for carrying out drug administration in a manner effective to treat vascular depression. The instructions will typically be written instructions on a package insert and/or on a label. The xanthine derivative and additional therapeutic agent may be formulated in any suitable pharmaceutical composition as described elsewhere herein.

Thus, in one embodiment, the invention relates to packaged kits for a subject to use in the treatment of vascular depression comprising: a) a first component comprising a xanthine derivative; and b) a second component comprising an SSRI. In one embodiment, xanthine derivative is a compound according to Formula I. In another embodiment, the xanthine derivative is pentoxifylline or propentofylline. In yet another embodiment, the SSRI is selected from the group consisting of citalopram, escitalopram, sertraline, paroxetine, fluoxetine, fluvoxamine, and dapoxetine. In another embodiment, the SSRI is citalopram or escitalopram.

In another embodiment, the invention relates to packaged kits for a subject to use in the treatment of vascular depression comprising: a) a first component comprising a xanthine derivative; and b) a second component comprising an SNRI. In one embodiment, xanthine derivative is a compound according to Formula I. In another embodiment, the xanthine derivative is pentoxifylline or propentofylline. In yet another embodiment, the SNRI is selected from the group consisting of venlafaxine, desvenlafaxine, nefazodone, milnacipran, desipramine, and duloxetine. In another embodiment, the SNRI is venlafaxine.

In another embodiment, the invention relates to packaged kits for a subject to use in the treatment of vascular depression comprising: a) a first component comprising a xanthine derivative; and b) a second component comprising an agent that is used in the treatment of cerebrovascular disease. In one embodiment, xanthine derivative is a compound according to Formula I. In another embodiment, the xanthine derivative is pentoxifylline or propentofylline. In yet another embodiment, the agent that is used in the treatment of cerebrovascular disease is selected from the group consisting of anticholesterolemic and antiplatelet agents, free radical scavengers, calcium channel blockers, glutamate N-methyl-D-aspartic acid (NMDA) receptor antagonists, gangliosides, aminosteroids, and amphetamines.

In one embodiment, the first and second components of these kits are contained in the same pharmaceutical formulation. In another embodiment, the first and second components of these kits are contained in separate pharmaceutical formulations. Where the first and second components are contained in separate pharmaceutical formulations, the packaged kit may include instructions that include directions for carrying out drug administration of the first and second (and/or third) components sequentially or concurrently.

Chemical Definitions

While the following terms are believed to be well understood by one of ordinary skill in the art, the following definitions are set forth to facilitate explanation of the presently disclosed subject matter. Unless otherwise defined, all technical and scientific terms used herein have the same meaning as commonly understood by one of ordinary skill in the art to which this presently described subject matter belongs.

Throughout the specification and claims, a given chemical formula or name shall encompass all optical and stereoisomers, as well as racemic mixtures where such isomers and mixtures exist.

When the term “independently selected” is used, the substituents being referred to (e.g., R groups, such as groups R₁, R₂, and the like, or groups X₁ and X₂), can be identical or different. For example, both R₁ and R₂ can be substituted alkyls, or R₁ can be hydrogen and R₂ can be a substituted alkyl, and the like.

A named “R” or “X” group will generally have the structure that is recognized in the art as corresponding to a group having that name, unless specified otherwise herein. For the purposes of illustration, certain representative “R” and “X” groups as set forth above are defined below. These definitions are intended to supplement and illustrate, not preclude, the definitions that would be apparent to one of ordinary skill in the art upon review of the present disclosure.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, such as a 3- to 7-member substituted or unsubstituted cycloalkyl ring system, including one or more heteroatoms, which can be the same or different, and are selected from the group consisting of N, O, and S, and optionally can include one or more double bonds. The cycloheteroalkyl ring can be optionally fused to or otherwise attached to other cycloheteroalkyl rings and/or non-aromatic hydrocarbon rings. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The term “alkenyl” as used herein refers to a straight or branched hydrocarbon of a designed number of carbon atoms containing at least one carbon-carbon double bond. Examples of “alkenyl” include vinyl, allyl, 2-methyl-3-heptene, and the like.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The term “heteroaryl” refers to an aromatic ring system, such as, but not limited to a 5- or 6-member ring system, including one or more heteroatoms, which can be the same or different, and are selected from the group consisting of N, O, and S. The heteroaryl ring can be fused or otherwise attached to one or more heteroaryl rings, aromatic or non-aromatic hydrocarbon rings, or heterocycloalkyl rings. Representative heteroaryl ring systems include, but are not limited to, pyridyl, pyrimidyl, pyrrolyl, pyrazolyl, azolyl, oxazolyl, isoxazolyl, oxadiazolyl, thiazolyl, isothiazolyl, imidazolyl, furanyl, thienyl, quinolinyl, isoquinolinyl, indolinyl, indolyl, benzothienyl, benzothiazolyl, enzofuranyl, benzimidazolyl, benzisoxazolyl, benzopyrazolyl, triazolyl, tetrazolyl, and the like.

A structure represented generally by the formula, wherein the ring structure can be aromatic or non-aromatic: 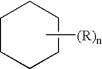
as used herein refers to a ring structure, for example, but not limited to a 3-carbon, a 4-carbon, a 5-carbon, a 6-carbon, and the like, aliphatic and/or aromatic cyclic compound, including a saturated ring structure, a partially saturated ring structure, and an unsaturated ring structure as defined herein, comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure above where n is 0 to 2 would comprise compound groups including, but not limited to: 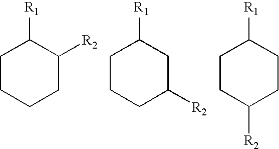
and the like.

A dashed line representing a bond in a cyclic ring structure indicates that the bond can be either present or absent in the ring. That is, a dashed line representing a bond in a cyclic ring structure indicates that the ring structure includes a saturated ring structure, a partially saturated ring structure, and an unsaturated ring structure.

When a named atom of an aromatic ring or a heterocyclic aromatic ring is defined as being “absent,” the named atom is replaced by a direct bond.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Alkoxyl” refers to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

The term “alkyl-thio-alkyl” as used herein refers to an alkyl-S-alkyl thioether, for example, a methylthiomethyl or a methylthioethyl group.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl. “Aryloxycarbonyl” refers to an aryl-O—CO— group. Exemplary aryloxycarbonyl groups include phenoxy- and naphthoxy-carbonyl. “Aralkoxycarbonyl” refers to an aralkyl-O—CO— group. An exemplary aralkoxycarbonyl group is benzyloxycarbonyl.

“Carbamoyl” refers to an H₂N—CO— group. “Alkylcarbamoyl” refers to a R′RN—CO— group wherein one of R and R′ is hydrogen and the other of R and R′ is alkyl and/or substituted alkyl as previously described. “Dialkylcarbamoyl” refers to a R′RN—CO— group wherein each of R and R′ is independently alkyl and/or substituted alkyl as previously described.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

The term “alkylamino” refers to an —NHR group wherein R is an alkyl group and/or a substituted alkyl group as previously described. Exemplary alkylamino groups include methylamino, ethylamino, and the like.

“Dialkylamino” refers to an —NRR′ group wherein each of R and R′ is independently an alkyl group and/or a substituted alkyl group as previously described. Exemplary dialkylamino groups include ethylmethylamino, dimethylamino, and diethylamino.

“Acylamino” refers to an acyl-NH— group wherein acyl is as previously described. “Aroylamino” refers to an aroyl-NH— group wherein aroyl is as previously described.

The term “carbonyl” refers to the —(C═O)— group.

The term “carboxyl” refers to the —COOH group.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄ group.

As used herein, an “analog” refers to a chemical compound in which one or more individual atoms or functional groups of a parent compound have been replaced, either with a different atom or with a different functional group. For example, thiophene is an analog of furan, in which the oxygen atom of the five-membered ring is replaced by a sulfur atom.

As used herein, a “derivative” refers to a chemical compound which is derived from or obtained from a parent compound and contains essential elements of the parent compound but typically has one or more different functional groups. Such functional groups can be added to a parent compound, for example, to improve the molecule's solubility, absorption, biological half life, and the like, or to decrease the toxicity of the molecule, eliminate or attenuate any undesirable side effect of the molecule, and the like. An example of a derivative is an ester or amide of a parent compound having a carboxylic acid functional group.

The following examples are offered by way of illustration and not by way of limitation.

### EXPERIMENTAL

### Example 1

### Pentoxifylline and Propentofylline Treatment of Vascular Depression

These case studies investigate the effects of pentoxifylline and propentofylline in one or more elderly patients (>60 years of age) with a diagnosis of vascular depression (assessed by Alexopolis criteria or MRI as described elsewhere herein, or for elderly patients who had previously failed to respond to standard antidepressant therapy). Patients are evaluated at baseline and at regular intervals for a period of months. They are evaluated according to the Hamilton Depressive Rating Scale (HDRS; Hamilton, J. Neurol. Neurosurg. Psychiatry 23:56-62 (1960)). Patients are excluded if their HDRS is ≦15.

In one group, pentoxifylline is administered orally in a dose of 400 mg three times a day. In another group, propentofylline is administered orally in a dose of 200 mg three times a day.

Results: HDRS scores are expected to decrease after several months in subjects receiving either pentoxifylline or propentofylline. Pentoxifylline or propentofylline therapy is expected to significantly improve depressive symptoms and be well tolerated in patients with vascular depression.

### Example 2

### Combination Treatment of Vascular Depression With SSRIs or SNRIs

These case studies investigate the effects of a combination treatment of pentoxifylline or propentofylline in combination with an SSRI or SNRI in one or more elderly patients with a diagnosis of vascular depression. Methods for the assessment of patients and for the administration of pentoxifylline or propentofylline are as described in Example 1. However, in addition to pentoxifylline or propentofylline, subjects are also administered escitalopram, citalopram, or venlafaxine. Escitalopram is administered orally in a dose of 10 mg once daily. Citalopram is administered orally in a dose of 20 mg once daily initially for one week and increasing to 40 mg once a day thereafter.

The individuals are monitored for a period of months and evaluated as described in Example 1.

Results: HDRS scores are expected to decrease after several months in subjects receiving either pentoxifylline or propentofylline in combination with either escitalopram, citalopram, or venlafaxine. These combination therapies are expected to significantly improve depressive symptoms and be well tolerated in patients with vascular depression.

### Example 3

### Pentoxifylline and Propentofylline Add-On Treatment of Vascular Depression

These case studies investigate the effects of pentoxifylline and propentofylline in one or more elderly patients (>60 years of age) with a diagnosis of vascular depression and who are currently being treated with an SSRI and/or SNRI.

Patients are evaluated at baseline and at regular intervals for a period of months. They are evaluated according to the Hamilton Depressive Rating Scale (HDRS; Hamilton, J. Neurol. Neurosurg. Psychiatry 23:56-62 (1960)).

In one group, pentoxifylline is administered orally in a dose of 400 mg three times a day to augment their SSRI and/or SNRI treatment regimen. In another group, propentofylline is administered orally in a dose of 200 mg three times a day to augment their SSRI and/or SNRI treatment regimen.

Results: HDRS scores are expected to decrease after several months in subjects receiving either pentoxifylline or propentofylline in addition to their existing SSRI and/or SNRI. Pentoxifylline or propentofylline therapy is expected to significantly improve depressive symptoms be well tolerated in patients with vascular depression.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for treating vascular depression in a subject in need thereof comprising administering to said subject a therapeutically effective amount of a xanthine derivative.

2. The method of claim 1, wherein said xanthine derivative is a compound according to Formula I 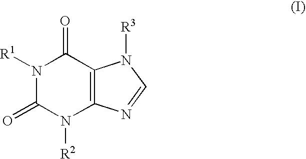
or a physiologically tolerable salt of the compounds of the Formula I, where R¹ is
a) an oxoalkyl group having 3 to 8 carbon atoms, whose carbon chain can be straight-chain or branched;
b) a hydroxyalkyl group having 1 to 8 carbon atoms, whose carbon chain can be straight-chain or branched and whose hydroxyl group is a primary, secondary or tertiary alcohol function; or
c) an alkyl group having 1 to 6 carbon atoms, whose carbon chain can be straight-chain or branched;
where R² is
a) a hydrogen atom; or
b) an alkyl group having 1 to 4 carbon atoms, whose carbon chain can be straight-chain or branched; and
where R³ is
a) a hydrogen atom;
b) an alkyl group having 1 to 6 carbon atoms, whose carbon chain can be straight-chain or branched;
c) an alkyl group having 1 to 6 carbon atoms, whose carbon chain is interrupted by an oxygen atom; or
d) an oxoalkyl group having 3 to 8 carbon atoms, whose carbon chain can be straight-chain or branched.

3. The method of claim 2, wherein
R¹ is an oxoalkyl group having 4 to 6 carbon atoms, whose carbon chain is straight-chain, or an alkyl group having 3 to 6 carbon atoms;
R² is an alkyl group having 1 to 4 carbon atoms; and
R³ is an alkyl group having 1 to 4 carbon atoms or an oxoalkyl having 3 to 6 carbon atoms.

4. The method of claim 3, wherein said compound of Formula I is 1-(5-oxohexyl)-3,7-dimethylxanthine(pentoxifylline) or 1-(5-oxohexyl)-3-methyl-7-n-propylxanthine(propentofylline).

5. The method of claim 4, wherein said compound of Formula I is 1-(5-oxohexyl)-3,7-dimethylxanthine(pentoxifylline) and is administered to said subject in a total daily dosage of between about 400 mg and about 1200 mg.

6. The method of claim 5, wherein 1-(5-oxohexyl)-3,7-dimethylxanthine(pentoxifylline) is administered to said subject in a dosage of about 400 mg three times a day.

7. The method of claim 4, wherein said compound of Formula I is 1-(5-oxohexyl)-3-methyl-7-n-propylxanthine(propentofylline) and is administered to said subject in a total daily dosage of between about 200 mg and about 600 mg.

8. The method of claim 7, wherein 1-(5-oxohexyl)-3-methyl-7-n-propylxanthine(propentofylline) is administered to said subject in a dosage of about 200 mg three times a day.

9. A method for treating vascular depression in a subject in need thereof comprising administering to said subject a therapeutically effective amount of a xanthine derivative in combination with an additional therapeutic agent selected from the group consisting of a selective serotonin reuptake inhibitor (SSRI), a serotonin-norepinephrine reuptake inhibitor (SNRI), and a drug used in the treatment of cerebrovascular disease.

10. The method of claim 9, wherein said xanthine derivative is a compound according to Formula I 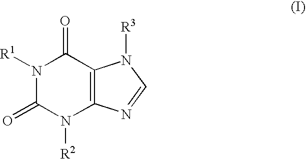
or a physiologically tolerable salt of the compounds of the Formula I, where R¹ is
a) an oxoalkyl group having 3 to 8 carbon atoms, whose carbon chain can be straight-chain or branched;
b) a hydroxyalkyl group having 1 to 8 carbon atoms, whose carbon chain can be straight-chain or branched and whose hydroxyl group is a primary, secondary or tertiary alcohol function; or
c) an alkyl group having 1 to 6 carbon atoms, whose carbon chain can be straight-chain or branched;
where R² is
a) a hydrogen atom; or
b) an alkyl group having 1 to 4 carbon atoms, whose carbon chain can be straight-chain or branched; and
where R³ is
a) a hydrogen atom;
b) an alkyl group having 1 to 6 carbon atoms, whose carbon chain can be straight-chain or branched;
c) an alkyl group having 1 to 6 carbon atoms, whose carbon chain is interrupted by an oxygen atom; or
d) an oxoalkyl group having 3 to 8 carbon atoms, whose carbon chain can be straight-chain or branched.

11. The method of claim 10, wherein
R¹ is an oxoalkyl group having 4 to 6 carbon atoms, whose carbon chain is straight-chain, or an alkyl group having 3 to 6 carbon atoms;
R² is an alkyl group having 1 to 4 carbon atoms; and
R³ is an alkyl group having 1 to 4 carbon atoms or an oxoalkyl having 3 to 6 carbon atoms.

12. The method of claim 11, wherein said compound of Formula I is 1-(5-oxohexyl)-3,7-dimethylxanthine(pentoxifylline) or 1-(5-oxohexyl)-3-methyl-7-n-propylxanthine(propentofylline).

13. The method of claim 9, wherein said additional therapeutic agent is an SSRI selected from the group consisting of citalopram, escitalopram, sertraline, paroxetine, fluoxetine, fluvoxamine, and dapoxetine.

14. The method of claim 13, wherein said SSRI is citalopram or escitalopram.

15. The method of claim 9, wherein said additional therapeutic agent is an SNRI is selected from the group consisting of venlafaxine, desvenlafaxine, nefazodone, milnacipran, desipramine, and duloxetine.

16. The method of claim 15, wherein said SNRI is venlafaxine.

17. A pharmaceutical composition comprising a xanthine derivative in combination with an additional therapeutic agent in therapeutically effective amounts for treating vascular depression in a subject in need thereof, wherein said additional therapeutic agent is selected from the group consisting of a selective serotonin reuptake inhibitor (SSRI), a serotonin-norepinephrine reuptake inhibitor (SNRI), and a drug used in the treatment of cerebrovascular disease, wherein said composition further comprises a pharmaceutically acceptable carrier.

18. The pharmaceutical composition of claim 17, wherein said xanthine derivative is a compound according to Formula I 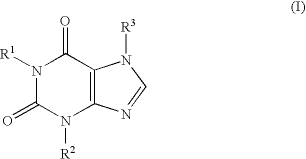
or a physiologically tolerable salt of the compounds of the Formula I, where R¹ is
a) an oxoalkyl group having 3 to 8 carbon atoms, whose carbon chain can be straight-chain or branched;
b) a hydroxyalkyl group having 1 to 8 carbon atoms, whose carbon chain can be straight-chain or branched and whose hydroxyl group is a primary, secondary or tertiary alcohol function; or
c) an alkyl group having 1 to 6 carbon atoms, whose carbon chain can be straight-chain or branched;
where R² is
a) a hydrogen atom; or
b) an alkyl group having 1 to 4 carbon atoms, whose carbon chain can be straight-chain or branched; and
where R³ is
a) a hydrogen atom;
b) an alkyl group having 1 to 6 carbon atoms, whose carbon chain can be straight-chain or branched;
c) an alkyl group having 1 to 6 carbon atoms, whose carbon chain is interrupted by an oxygen atom; or
d) an oxoalkyl group having 3 to 8 carbon atoms, whose carbon chain can be straight-chain or branched.

19. The pharmaceutical composition of claim 18, wherein
R¹ is an oxoalkyl group having 4 to 6 carbon atoms, whose carbon chain is straight-chain, or an alkyl group having 3 to 6 carbon atoms;
R² is an alkyl group having 1 to 4 carbon atoms; and
R³ is an alkyl group having 1 to 4 carbon atoms or an oxoalkyl having 3 to 6 carbon atoms.

20. The pharmaceutical composition of claim 19, wherein said compound of Formula I is 1-(5-oxohexyl)-3,7-dimethylxanthine(pentoxifylline) or 1-(5-oxohexyl)-3-methyl-7-n-propylxanthine(propentofylline).

21. The pharmaceutical composition of claim 17, wherein said additional therapeutic agent is an SSRI selected from the group consisting of citalopram, escitalopram, sertraline, paroxetine, fluoxetine, fluvoxamine, and dapoxetine.

22. The pharmaceutical composition of claim 21, wherein said SSRI is citalopram or escitalopram.

23. The pharmaceutical composition of claim 17, wherein said additional therapeutic agent is an SNRI is selected from the group consisting of venlafaxine, desvenlafaxine, nefazodone, milnacipran, desipramine, and duloxetine.

24. The pharmaceutical composition of claim 23, wherein said SNRI is venlafaxine.

25. A packaged kit for use in the treatment of vascular depression in a subject in need thereof comprising:
a) a first component comprising a xanthine derivative;
b) a second component comprising an additional therapeutic agent selected from the group consisting of a selective serotonin reuptake inhibitor (SSRI), a serotonin-norepinephrine reuptake inhibitor (SNRI), and a drug used in the treatment of cerebrovascular disease; and
c) instructions for carrying out drug administration of said first and second components in a manner effective to treat said vascular depression.
